(19) United States
(12) Patent Application Publication
(10) Pub. No.: US 2009/0287049 A1
(43) Pub. Date: Nov. 19, 2009

(54) Access Systems Including Collapsible Port Body For Intra-Abdominal Surgery

(76) Inventors: Donald K. Jones, Dripping Springs, TX (US); Dennis L. McWilliams, Austin, TX (US); Vladimir Mitelberg, Austin, TX (US); Brett E. Naglreiter, Austin, TX (US)

Correspondence Address: GORDON & JACOBSON, P.C., 60 LONG RIDGE ROAD, SUITE 407, STAMFORD, CT, 06902, US

(21) Appl. No.: 12/121,478

(22) Filed: May 15, 2008

(51) Int. Cl. A61B 1/01

(52) U.S. Cl. 600/115; 600/114

(57) ABSTRACT

A natural orifice translucent endoscopic surgery (NOTES) comprises a thin, flexible body preferably made from a lubricious film. The port body also includes a coupler to removably attach the port body to an endoscope received through the port body. According to embodiments, the port body also includes a structurally modifiable port body that can be modified while the port body is within the patient. In one embodiment, the port body is constructed with a channel that preferably coils around the port body. The channel can be inflated and deflated to provide relative degrees of rigidity to the port body.

### BACKGROUND OF THE INVENTION

1. Field of the Invention

The present invention relates to access systems for providing secure access to the abdominal cavity through a wall of a body cavity reached through a natural orifice, and methods of performing intra-abdominal surgical procedures through such an access system using an endoscope.

2. State of the Art

The field of gastrointestinal endoscopy has for many years been limited to diagnostic and therapeutic techniques to observe, modify and remove tissues located in the digestive tract. Only recently have there been efforts to expand gastrointestinal endoscopic surgery to within the peritoneal cavity to remove large tissue masses such as the appendix and gallbladder. Generally, in these newer procedures, a natural orifice translucent endoscopic surgery (NOTES) access system is used to provide secure access to the peritoneal cavity through the stomach or another natural orifice. However, there are still significant limitations to the present techniques for manipulating and removing masses of tissue on current access systems.

### SUMMARY OF THE INVENTION

According to embodiments of the invention, a natural orifice translucent endoscopic surgery access system is provided for enabling and facilitating access to the abdominal cavity through an intragastric or transvaginal approach. In each embodiment, the access system includes a structurally modifiable port body, a proximal handle, an endoscope attachment means at a distal end of the port body to attach the port body to an endoscope, and a securing system at a distal end of the port body that secures the port body within a hole provided in a wall of a body cavity accessible via a natural orifice.

The port body comprises a thin, flexible body preferably made from a lubricious film. The endoscope attachment means preferably includes an elastic collar or an inflatable collar. The securing system preferably includes individually expandable proximal and distal cuffs, permitting fixation of the cuffs on opposite sides of a wall separating a natural orifice from the peritoneal cavity.

According to one embodiment, the port body is constructed with a channel that preferably coils around the port body. The channel can be inflated and deflated to provide relative degrees of rigidity to the port body. In a preferred initial configuration, the channel in the port body is deflated (to provide the port with increased flexibility), and the port body is advanced over an endoscope and secured at its distal end to the endoscope.

For use in an intragastric procedure, the endoscope is used to advance the port body through the gastric interior. Once the port body and the endoscope enter the gastric interior, the port channel is inflated to stiffen the port body. A surgical cutting tool is delivered through the endoscope working channel to incise the gastric wall. A dilation balloon may then be used to enlarge the incision to a size sufficient for the port to enter the peritoneal space. The distal end of the port body is passed through the incision into the peritoneal cavity. The proximal cuff is inflated in the gastric interior, followed by inflation of the distal cuff in the peritoneal cuff to secure the port around the gastric wall. Secured access is provided through the port to the peritoneum for a surgical procedure.

According to another embodiment, the endoscope attachment means is an inflatable internal collar. In a preferred initial configuration of the second embodiment, the port body is provided over an endoscope and the internal collar is inflated to secure the port to the endoscope.

For use in an intragastric procedure, the endoscope is used to advance the port body through the gastric interior. Once the port and the endoscope enter the gastric interior, a surgical cutting tool is delivered to incise the gastric wall. A dilation balloon may then be used to enlarge the incision to a size sufficient for the port to enter the peritoneal space. After that, the distal end of the port is passed through the incision into the peritoneal cavity. The proximal cuff is inflated in the gastric interior, followed by inflation of the distal cuff in the peritoneal cavity to secure the port around the gastric wall. Then, the internal collar is deflated releasing the endoscope from the port. The endoscope is then withdrawn from the patient. Once the endoscope is removed, the port provides secured access to the peritoneum for a surgical procedure. An additional inflatable channel may be provided about the port body to control rigidity of the port from a proximal handle.

Additional objects and advantages of the invention will become apparent to those skilled in the art upon reference to the detailed description taken in conjunction with the provided figures.

### BRIEF DESCRIPTION OF THE DRAWINGS

FIG. 1 is a longitudinal section view of an access system according to a first embodiment of the invention.

FIG. 2 is a schematic illustration of a distal end of the access system of FIG. 1.

FIG. 3 is illustrates the access system and endoscope inserted into a patient.

FIGS. 4 through 12 illustrate a method according to the invention.

FIG. 13 is a longitudinal section view of an access system according to a second embodiment of the invention.

### DETAILED DESCRIPTION OF THE PREFERRED EMBODIMENTS

A natural orifice translucent endoscopic surgery (NOTES) access system is provided for enabling and facilitating access to the peritoneal cavity through an anatomical wall, the anatomical wall separating the peritoneal cavity and a natural orifice accessible body cavity. While the invention is primarily described with respect to a through-the-esophagus transgastric approach for such surgery, where the body cavity is the stomach and the anatomical wall is the gastric or stomach wall, the systems and methods described herein are equally applicable to procedures performed transanally, wherein the body cavity is the colon and the anatomic wall is the colon wall, and transvaginally wherein the body cavity is the vagina and the anatomic wall is the vaginal wall.

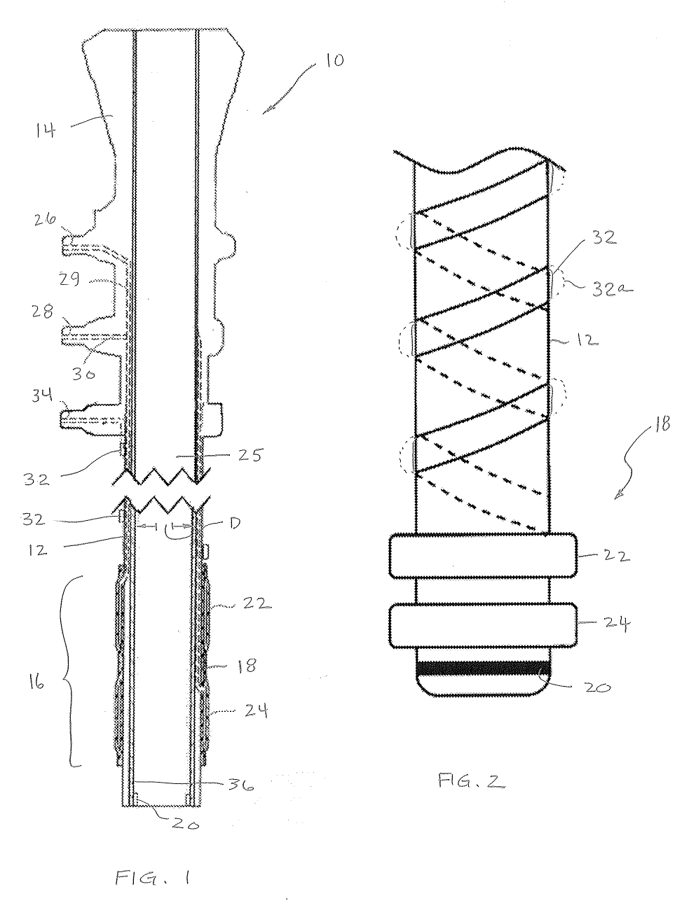

Turning now to FIGS. 1 and 2, the access system 10 includes a structurally modifiable port body 12, a proximal handle 14, a gastric wall securing system, generally 16, at the distal end 18 of the port body that temporarily secures the port body within a hole in the gastric wall, and an endoscope attachment means 20 at a distal end 18 of the port body to attach the port body to an endoscope.

In a preferred embodiment, the gastric wall securing system 16 includes proximal and distal inflatable cuffs 22, 24 provided on an external portion of the distal end 18 of the port body 12. The cuffs 22, 24 are in communication with respective valved injection ports 26, 28 at the handle 14 through air channels 29, 30 to permit individual pressurization with a fluid, e.g., air, to fixate the cuffs on opposite sides of the gastric wall. This secures the port body 12 to the gastric wall and provides a seal between the intragastric space and the peritoneal cavity.

The port body 12 distal of the handle 14 is a collapsible tube with a lumen 25 having a diameter D. The port body 12 has length in the range of 20 to 60 inches, with a preferred range of 30 to 45 inches; and a lumen diameter D in the range of about 5 to 18 mm. The port body length is sufficient to extend from a patient's mouth to a patient's stomach or from any other natural orifice to a body cavity accessible therefrom.

The port body 12 is constructed of a thin film permitting the body to be longitudinally and laterally flexible. The port body 12 is preferably made from a lubricious polymeric film comprised of a polyester, a polyolefin, a fluoropolymer, or blends thereof. By way of example, and not by limitation, polyethylene, polytetrafluoroethylene (PTFE), polyethylene terephthalate (PET) or polyvinylidene fluoride (PVDF) can be used. These materials facilitate movement of the port body through the natural orifice and instruments and materials through the lumen 25 of the port body. Also, an elastopolymer film can be used, permitting temporary changes in diameter D of lumen 25 along the length of the port body when the port body is subject to internal radial force. This permits passage of materials larger than diameter D through lumen 25. It is, however, understood that the anatomy (e.g., esophagus) may be the limiting factor in the maximum permitted size of material and instruments through the resilient port body 12.

According to a preferred aspect of this embodiment of the invention, the port body 12 is constructed with or otherwise provided with an channel 32 that can be inflated or deflated to alter the rigidity of the port. The channel 32 preferably coils around the port body 12. The channel 32 can be integrated within the port body, e.g., defined between two layers of the film comprising the port body, or a separate tubular construct bonded to the inner or outer surfaces of the film or between layers of the film. The handle 14 is provided with a port 34 that communicates with the channel 32 to control inflation of the channel with a fluid, e.g., air, to inflate the channel. Multiple channels can be used.

Upon inflation of the channel 32, the channel expands to state 32a as shown by broken line in FIG. 2. The expansion of channel 32 increase at least one of the longitudinal and lateral rigidity of the port body 12, and preferably both of the longitudinal and lateral rigidity are increased. Likewise, prior to inflation or upon deflation of the channel, the rigidity of the port body 12 is relatively lower than in the inflated state. By controlling the inflation and deflation of the channel 32 (and the pressure of fluid therein), the port body 12 can be provided with a determined degree of flexibility or rigidity.

With the rigidity of the port body 12 controllable throughout a procedure, as described in more detail below, specific advantage is provided during port body insertion and removal. With the port body deflated, the highly flexible port body can accommodated through the natural orifice (e.g., esophagus) while providing protection of the natural orifice lining (e.g., esophageal lining). With the port body channel 32 inflated, the port body 12 provides a well-defined secure channel through which an intragastric surgical procedure can be conducted.

Further, the flexible port body 12 provides adaptability for removing tissue en masse therethrough. Where a resilient film is used, constraints presented by prior art ports having a fixed diameter along their length and high lateral and/or longitudinally rigidity throughout a procedure are eliminated.

Referring to FIGS. 1 and 2, the endoscope attachment means is an elastic collar 20 provided at the distal end 18 of the port body 12. The elastic collar 20 is preferably provided on the inner surface 36 of the port body 12 and attaches the port body to an endoscope.

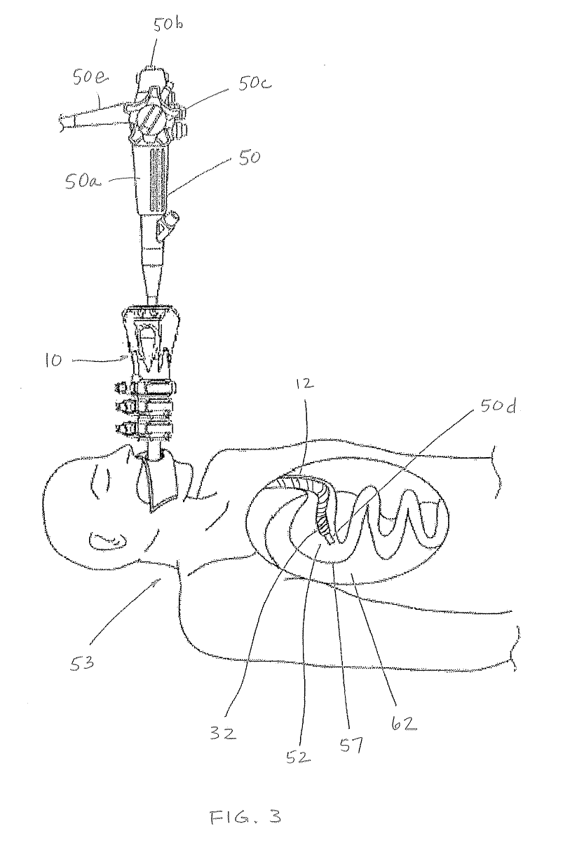

Referring to FIGS. 1 and 3, in a preferred initial configuration of access system 10, the channel 32 of the port body 12 is deflated (to provide the port with increased flexibility), and the port body is advanced over an endoscope 50 and secured to the endoscope at the elastic collar 20. The endoscope 50 includes a proximal handle 50a including access to a working channel 50b, a knob 50c to control steerability of the distal end 50d, and a monitor line out 50e for the imaging sensor/lens at its distal end 50d.

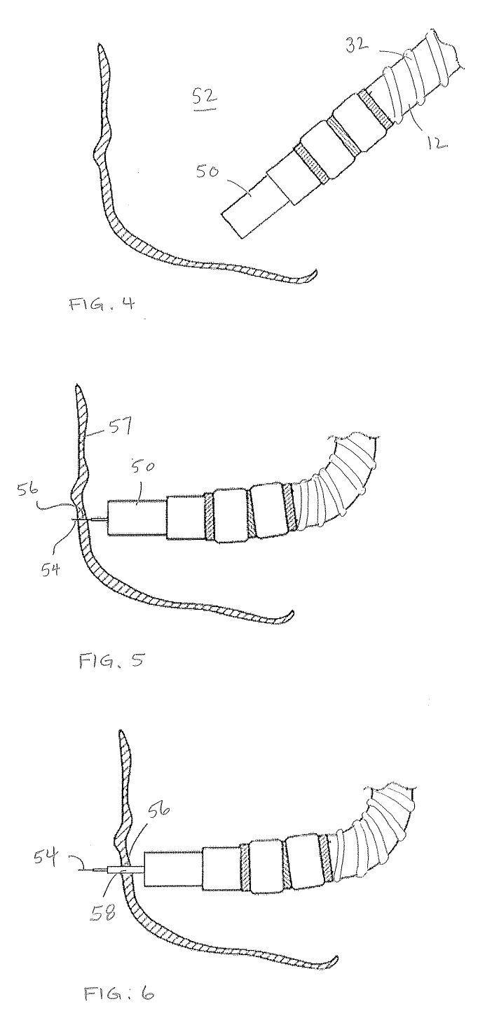

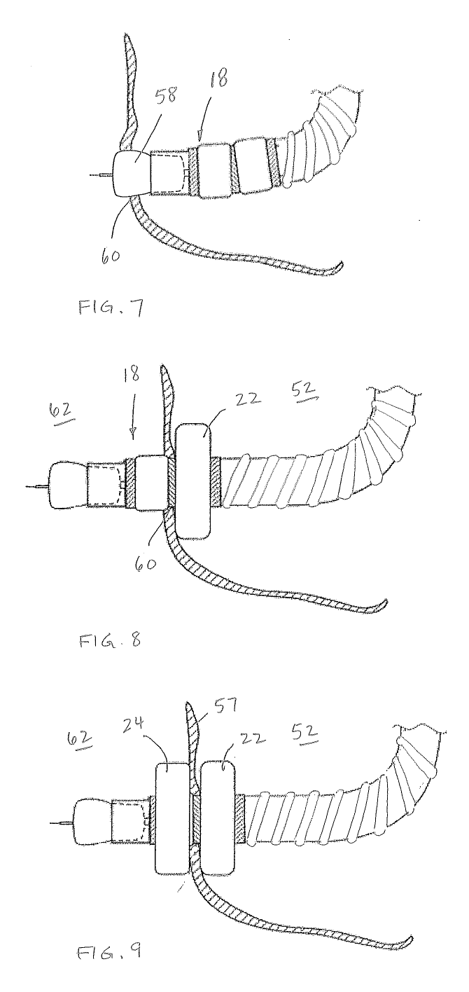

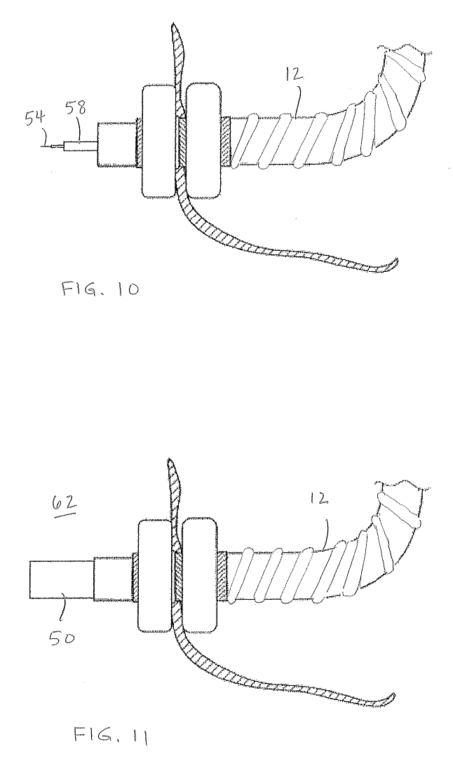

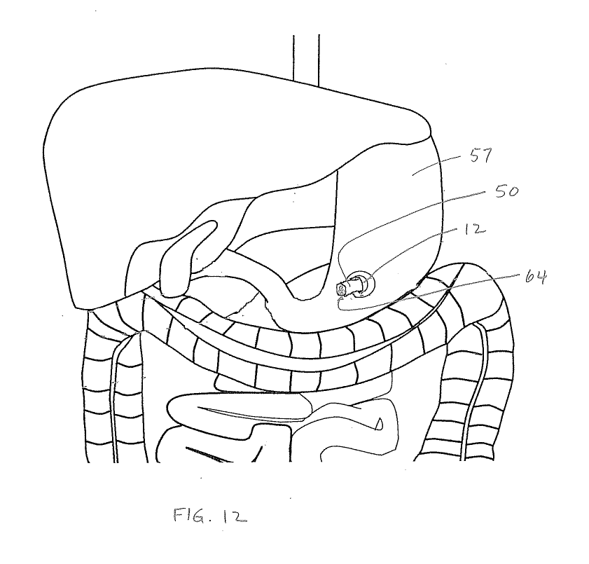

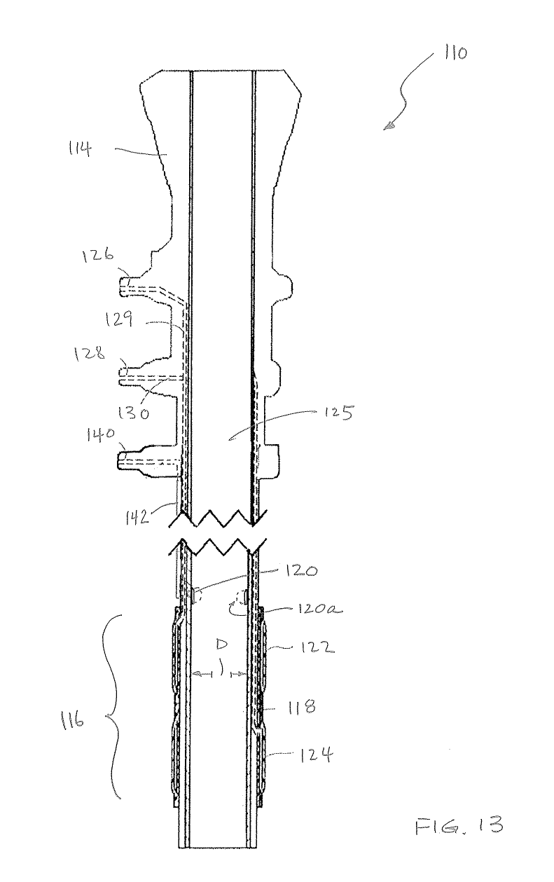

Then, in use, the endoscope 50 is used to maneuver the port body 12 into the gastric interior 52 (FIG. 3) of the patient 53. The lubricious quality of the film facilitates positioning the port body through the esophagus. Once the port body 12 and the endoscope 50 enter the gastric interior 52, the channel 32 is inflated to increase the rigidity of (i.e., stiffen) the port body 12 (FIG. 4). Inflation of the channel 32 also maintains the lumen 25 patent during the procedure (FIGS. 1 and 4). A surgical cutting tool 54 is delivered through a working channel of the endoscope 50 to create a piercing 56 in the gastric wall 57 (FIG. 5). A dilation balloon 58 may then be advanced into the piercing 56. The balloon 58 may be advanced over the cutting tool 54 to facilitate guiding it into the piercing (FIG. 6). The balloon 58 is then expanded to enlarge the piercing to a hole 60 of sufficient size to receive the distal end 18 of the port body (FIG. 7). The distal end 18 of the port body is passed through the hole 60 and into the peritoneal cavity 62 (FIG. 8). The proximal cuff 22 is inflated in the gastric interior 52, followed by inflation of the distal cuff 24 in the peritoneal cavity 62 to secure the port body around the gastric wall 57 (FIGS. 8 and 9). The balloon 58 is deflated and removed (FIG. 10), the cutting tool 54 is optionally removed, and the endoscope and other instruments may be advanced into the peritoneal cavity 62 through the access port 12 and lumen 64 of the endoscope 50 (FIGS. 11 and 12). Secured access is thereby provided through the port body 12 to the peritoneal cavity for a surgical procedure, including en masse tissue separation.

The port body can also be introduced transanally and up the colon to enter the peritoneal cavity at a location other than through the stomach. Further, while the access system can be used intragastrically, it can also similarly be used transvaginally to perform a surgical procedure. To that end, it is coupled at its distal end to an endoscope that is introduced transvaginally, introduced through a wall of the vagina into the abdominal cavity and secured in the vaginal wall to provide secure access for a surgical procedure. The rigidity of the port body of the access system is modifiable via controlled inflation and deflation of the channel of the port body.

Turning now to FIG. 13, another embodiment of the invention is shown. Access system 110 includes the port body 112, a handle 114, a gastric wall securing system 116 at the distal end 118 of the port body that secures the port body within a hole in the gastric wall, and an endoscope attachment means 120 at a distal end 118 of the port body to attach the port body to an endoscope.

The port body 112 is a collapsible tube with a lumen 125 having a diameter D. The port body 112 is constructed of a thin film permitting the port body to be longitudinally and laterally flexible. The port body 112 is preferably made from a lubricious polymeric film comprised of a polyester, a polyolefin, a fluoropolymer, elastopolymer, or blends thereof.

The gastric wall securing system 116 includes proximal and distal inflatable cuffs 122, 124 provided on an external portion of the distal end 118 of the port body 112. The cuffs 122, 124 are in communication with respective injection ports 126, 128 at the handle 114 through air channels 129, 130 to permit individual pressurization with a fluid, e.g., air, to fixate the cuffs on opposite sides of the gastric wall. This secures the distal end 118 of the port body 112 to the gastric wall and provides a seal between the intragastric space and the peritoneal cavity.

The endoscope attachment means includes an inflatable collar 120 preferably within the distal end 118 of the port body 112, shown inflated as 120a. A valved injection port 140 is provided at the handle 114, and fluid conduit 142 extends along the film from the port 140 to the collar 120. The conduit 142 can be integrated within the port body, e.g., defined between two layers of the film comprising the port body, or a separate tubular construct bonded to the inner or outer surfaces of the film or between layers of the film. The pressurization of the collar 120 (and coupling and decoupling of the access system to the endoscope) can be actuated from the handle 114.

An inflatable channel, similar to channel 26 (FIGS. 1-2), can also be provided to access system 110 to permit controlling the rigidity of the port body along with an associated fourth injection port and fluid conduit (not shown).

Access system 110 is used substantially the same of the access system 10. In a preferred initial configuration, the access system is fed over an endoscope and the collar 120 is inflated to engage the distal end 118 of the port body 112 to an endoscope. The endoscope is inserted into an anatomical cavity through a natural orifice and advanced according to methods described herein through a hole in a wall of the anatomical cavity. The cuffs 122, 124 are inflated to secure the distal end 118 of the port body relative to the anatomical wall and create a fluid tight seal between the anatomical cavity and the abdominal cavity. The collar 120 can then be deflated, and the endoscope maneuvered further into the abdominal cavity or withdrawn, as necessary. If the access system 110 is provided with an inflatable channel along port body length, such channel can be inflated to provide the port lumen 124 with increased rigidity. An access port is thereby provided into the abdominal cavity of the patient through a natural body orifice.

There have been described and illustrated herein several embodiments of an access system and methods of performing intra-abdominal surgery. While particular embodiments of the invention have been described, it is not intended that the invention be limited thereto, as it is intended that the invention be as broad in scope as the art will allow and that the specification be read likewise. Thus, while particular means of attaching the distal end of a port body to an endoscope have been described, it will be appreciated that means in addition to elastics and inflatable collars can be used as well. In addition, while a particular gastric wall securing system has been disclosed, it will be appreciated that other gastric wall securing system can be used as well, including mechanically expandable systems. Further, while particular types of instruments for the cutting and piercing tissue, and drawing a balloon from a natural orifice accessible body cavity to within an anatomical wall of the body cavity wall have been disclosed, it will be understood that other suitable instruments can be used as well. It will therefore be appreciated by those skilled in the art that yet other modifications could be made to the provided invention without deviating from its spirit and scope as claimed.

What is claimed is:

1. An access system for accessing a patient's peritoneal cavity from a body cavity accessible through a natural orifice, the peritoneal cavity and body cavity separated by an anatomical wall, said access system for use with an endoscope, said access system comprising:
a) a tubular member having a proximal portion and a distal portion with a distal end, and a length therebetween sufficient to extend from a patient's mouth to a patient's stomach, said tubular member formed of a collapsible lubricious film permitting said tubular member to be at least partially collapsed in length, said tubular member defining a lumen for receiving the endoscope therethrough;
b) an anatomical wall securing system at said distal portion of said tubular member that includes expandable structure that temporarily secures said distal portion of said tubular member within a hole in the anatomical wall;
c) a handle at said proximal portion of said tubular member for operating said securing system; and
d) a coupling element for coupling said distal end of said tubular member to the endoscope.

2. An access system according to claim 1, wherein:
said tubular member is longitudinally and laterally flexible.

3. An access system according to claim 2, wherein:
said film is a polyester.

4. An access system according to claim 2, wherein:
said film is a polyolefin.

5. An access system according to claim 2, wherein:
said film is a fluoropolymer.

6. An access system according to claim 2, wherein:
said film is a blend one more than one of a polyester, a polyolefin, and a fluoropolymers.

7. An access system according to claim 2, wherein:
said film is an elastopolymer.

8. An access system according to claim 2, wherein:
said coupling element is an elastic collar.

9. An access system according to claim 2, wherein:
said coupling element is an inflatable collar.

10. An access system according to claim 2, wherein:
said coupling element is provided at an interior of said tubular member.

11. An access system according to claim 2, wherein:
said coupling element is actuatable from said handle.

12. An access system according to claim 2, wherein:
said tubular member can be provided with a determined degree of rigidity.

13. An access system according to claim 2, further comprising:
an inflatable channel extending along said tubular member from said proximal handle to said distal end, wherein inflating said channel operates to decrease the flexibility of said tubular member, and deflating said channel operates to increase the flexibility of said tubular member.

14. An access system according to claim 13, wherein:
said channel coils about said tubular member.

15. An access system according to claim 12, wherein:
said channel extends along an exterior of said tubular member.

16. An access system according to claim 12, wherein:
said gastric wall securing system includes a proximal inflatable cuff and a distal inflatable cuff, both cuffs at said distal end of said tubular member, first and second injection ports provided at the handle and respective air channels to permit individual pressurization of said proximal and distal cuffs.

17. An access system for accessing a patient's peritoneal cavity from a body cavity accessible through a natural orifice, the peritoneal cavity and body cavity separated by an anatomical wall, said access system for use with an endoscope, said access system comprising:
a) a lubricious tubular member having a proximal portion and a distal portion with a distal end, and a length therebetween sufficient to extend from a patient's mouth to a patient's stomach, said tubular member having laterally flexibility and longitudinally collapsible, said tubular member defining a lumen for receiving the endoscope therethrough;
b) an anatomical wall securing system at said distal portion of said tubular member that includes expandable structure that temporarily secures said distal portion of said tubular member within a hole in the anatomical wall;
c) a handle at said proximal portion of said tubular member for operating said securing system; and
d) a coupling element for coupling said distal end of said tubular member to the endoscope.

18. An access system according to claim 17, further comprising:
an inflatable channel extending along said tubular member from said proximal handle to said distal end, wherein inflating said channel operates to decrease the flexibility of said tubular member, and deflating said channel operates to increase the flexibility of said tubular member.

19. An access system according to claim 18, wherein:
said channel coils about said tubular member.

20. An access system according to claim 18, wherein:
said channel extends along an exterior of said tubular member.

21. An access system according to claim 17, wherein:
said tubular member is comprised of a film material.

22. A system for accessing a patient's peritoneal cavity from a body cavity accessible through a natural orifice, the peritoneal cavity and body cavity separated by an anatomical wall, said assembly comprising:
a) a flexible endoscope including an optical imaging system and a working channel and having a length sufficient to extend from a patient's mouth to a patient's stomach, the endoscope having a proximal end with a proximal handle and a distal end; and
b) an access system for placement through the natural orifice of the patient and the body cavity accessible from the natural orifice, the access system including,
i) a tubular member having a proximal portion and a distal portion with a distal end, and a length therebetween sufficient to extend from a patient's mouth to a patient's stomach, said tubular member formed of a collapsible lubricious film permitting said tubular member to be at least partially collapsed in length, said tubular member defining a lumen in which the distal portion of the endoscope is received,
ii) a coupling element for coupling said distal portion of said tubular member to said distal portion of said endoscope,
iii) an anatomical wall securing system at said distal portion of said tubular member that includes expandable structure that temporarily secures said distal portion of said tubular member within a hole in the anatomical wall, and
iv) an access system handle at said proximal portion of said tubular member including a controller for said securing system.

23. A system according to claim 22, wherein:
said endoscope is steerable from said proximal handle.

24. A system according to claim 22, wherein:
said endoscope can be decoupled from said coupling element after said anatomical wall securing system has secured said tubular member relative to said anatomical wall and while said distal portions of said tubular member and said endoscope are within the patient.

25. A system according to claim 22, wherein:
said coupling element is an elastic collar.

26. A system according to claim 22, wherein:
said coupling element is an inflatable collar.

27. A system according to claim 22, wherein:
said coupling element is provided at an interior of said tubular member.

28. A system according to claim 22, wherein:
said film is one of a polyester, a polyolefin, a fluoropolymer, and a blend one more than one of a polyester, a polyolefin, and a fluoropolymer.

29. A method of intra-abdominal surgery within a peritoneal cavity of a patient, comprising:
a) introducing a natural orifice translucent endoscopic surgery (NOTES) access system into a body cavity accessible through a natural orifice, the access system having tubular member with a proximal end and a distal end, a handle at said proximal end, and a lumen for insertion of an endoscope therethrough; and
b) modifying the longitudinal and lateral rigidity of the tubular member from said handle while said distal end is within the patient.

30. A method according to claim 29, wherein:
said tubular member comprises a film structure.

31. A method according to claim 29, wherein:
said tubular member is provided with a fluid channel, and said modifying includes changing the amount of fluid within said fluid channel.

32. A method according to claim 29, further comprising:
before said modifying,
an inserting an endoscope through said lumen;
coupling said endoscope to said tubular member; and
inserting said endoscope and tubular member into the patient.

33. A method according to claim 32, further comprising:
after said modifying,
decoupling said endoscope from said tubular member; and
withdrawing said endoscope from said tubular member and said patient.

34. A method of intra-abdominal surgery within a peritoneal cavity of a patient, comprising:
a) introducing a natural orifice translucent endoscopic surgery (NOTES) access system into a body cavity accessible through a natural orifice, the access system having tubular member with a proximal end and a distal end, a handle at said proximal end, and a lumen therethrough;
b) inserting an endoscope through said lumen;
c) coupling said endoscope to said tubular member;
d) inserting said endoscope and tubular member into the patient;
e) inserting said endoscope and said tubular member through an anatomical wall separating the body cavity from the peritoneal cavity;
f) coupling said tubular member within the anatomical wall;
g) decoupling said endoscope from said tubular member and removing said endoscope from said tubular member sand said patient;
h) decoupling said tubular member from said anatomical wall;
i) withdrawing said tubular member said patient.

35. A method according to claim 34, further comprising:
inserting instrument through a working channel of said endoscope prior to removing said endoscope.

36. A method according to claim 34, wherein:
said tubular member includes an elastic member, and
said coupling said endoscope to said tubular member includes frictionally engaging an elastic member in said tubular member about said endoscope, and
said decoupling said endoscope from said tubular member includes frictionally disengaging said endoscope from said elastic member.

37. A method according to claim 34, wherein:
said tubular member includes an inflatable collar, and
said coupling said endoscope to said tubular member includes inflating said collar to engage said tubular member about said endoscope, and
said decoupling said endoscope from said tubular member includes deflating said collar to release said endoscope from said collar.
